(19) United States
(12) Patent Application Publication
(10) Pub. No.: US 2004/0143171 A1
(43) Pub. Date: Jul. 22, 2004

(54) Method for generating patient medication treatment recommendations

(76) Inventor: Ralph F. Kalies, Pickett, WI (US)

Correspondence Address: THOMPSON HINE L.L.P., 2000 COURTHOUSE PLAZA , N.E., 10 WEST SECOND STREET, DAYTON, OH, 45402, US

(21) Appl. No.: 10/756,043

(22) Filed: Jan. 13, 2004

Related U.S. Application Data
(60) Provisional application No. 60/439,718, filed on Jan. 13, 2003.

(51) Int. Cl. A61B 5/00

(52) U.S. Cl. 600/300; 128/925

(57) ABSTRACT

A method for gathering, organizing, and assessing patient information, then providing feedback to the patient and authorized caregivers. The patient provides health status and medication information to a processing center via an electronic communication network. The data may be encrypted before transmission for further protection. The patient's information is collected, organized, and stored in a data warehouse. Once collected, the patient information can be analyzed by a drug therapy analysis engine which reviews the patient's medications and health status in accordance with previously established medical practices and standards to determine whether the patient is receiving the optimum types and dosages of medication. Further, the drug therapy analysis engine can check for potential adverse side effects or drug interactions, given that patient's medical history and particular drug regimen. Security methods may be employed to prevent unauthorized parties from viewing or tampering with the data.

This application claims priority to U.S. provisional application No. 60/439,718, filed Dec. 13, 2003, the contents of which are hereby incorporated by reference.

### FIELD

This invention relates to a method for monitoring patient therapy. Specifically, the invention relates to a method for gathering patient-specific medical data, compiling and assessing the data, and providing real-time feedback to the patient, the patient's health care providers, and other authorized users by means of an electronic communication network such as an intranet or the Internet. The feedback may include advisories such as potential drug interactions and/or side effects.

### BACKGROUND

Managed Care Organizations (“MCOs”) work to minimize the cost of health care for their subscribers through a variety of means, including volume purchases, quality control, and negotiated fees. MCOs also have a high level of interest in ensuring that patients receive proper and cost-effective medical treatment in order to achieve a satisfactory patient outcome while controlling costs.

Patients subscribing to MCOs are often diagnosed as having a variety of medical conditions, often by a number of different health care providers. This is particularly prevalent among non-institutionalized ambulatory patients, in contrast to those patients who are residents of a health care or nursing home facility. Such ambulatory patients frequently visit a number of medical specialists for treatment. As a result, these patients may receive medications that can cause an adverse reaction when taken in combination with medications previously prescribed for other conditions. Since medical personnel and pharmacists are not always privy to their patients' complete treatment regimen, and because prescription data may not always be adjudicated by a common MCO, there is a significant risk that such patients will eventually suffer harm by taking such conflicting medicines. For example, it has been noted by the health care industry that hospital admissions among the elderly due to adverse consequences and therapeutic failures of drug therapy are six times that of the general population. This accounts for a great deal of expense that could be avoided if the ambulatory patients' medical history could be scrutinized by the appropriate personnel in making a diagnosis and prior to dispensing medication or prescribing other therapy.

It must also be recognized that new and more effective pharmacological and medical treatments are constantly being developed that offer the possibility of an increased life span and/or improved quality of life. However, the increasing availability of these new treatments increases the attendant cost and potential drug interaction risk. As the universe of available drugs and medical treatments expands, the possibility of an unintended result or reaction also increases, subjecting the patient to greater health risk while seeking relief from an ailment.

There is also a desire to improve the cost-effectiveness of health care. This problem is of growing concern as the median age of the population rises and the correspondingly increased demand for health care continues to strain the financial viability of such healthcare resources as medical insurance plans. As a result, insurers and their insured are both placed at economic risk.

There is a need for a method to optimize patients' drug therapy regimens and to reduce the incidence of avoidable drug interactions. There is also a need for an improved means for monitoring the effects of new medications and treatments on patients. There is a still further need to increase the efficiency of providing quality healthcare in order to curb the rate of inflation of healthcare costs.

### Summary

According to the present invention, a method is disclosed for collecting, organizing, and assessing patient information, then providing feedback to the patient and authorized caregivers. The patient provides health status, clinical information and medication information to a processing center for the MCO. It is preferable that the patient's information be collected and organized in a predetermined electronic format, and be periodically updated by the patient in order to maintain currency. This information may be transmitted by the patient to the processing center via an electronic communication network such as an intranet or the Internet. Security methods such as usernames and passwords may be employed to prevent unauthorized parties from viewing or tampering with the data. The data may also be encrypted via conventional means prior to transmission for further protection.

Patient-specific medical, clinical and demographic information may be collected, organized, and stored in a “data warehouse.” A data warehouse is an electronic database wherein large quantities of related data from many computer operating systems such as, for example, UNIX, Microsoft MS-DOS, Windows NT, Windows 2000, Linux, IBM OS/2, the Apple Macintosh Operating System, DEC VMS and TOPS-20 are merged into a single database to provide an integrated informative view based on logical queries. The data warehouse is a valuable tool that can provide information for use in a wide variety of therapeutic, statistical, and economic analyses to aid the MCO medical and business staffs in making health care and business related decisions. The data warehouse can also provide feedback information for analysis of the impact of prior decisions on patient outcomes, facilitating improvements in patient care and operational efficiency by reducing the cost of medical care.

Once collected, the patient information can be utilized for two primary functions. First, the data can be automatically analyzed according to the present invention by a drug therapy analysis engine that reviews the patient's medications and health status in accordance with previously established medical practices and standards to determine whether the patient is receiving the optimum types and dosages of medication. Further, the drug therapy analysis engine can check for potential adverse side effects or drug interactions, given a patient's medical history and current drug treatment regimen. After completing the analysis, the drug therapy analysis engine can automatically generate a report containing the results of the analysis and provide recommendations for optimizing the patient's drug therapy. The processing center can electronically transmit the report to the patient or their family for review and further action, such as an informed consultation with the patient's health care providers. The report may also be sent directly to the patient's authorized health care providers for review and further action. The process of patient reporting and feedback is periodic, allowing for on-going monitoring, assessment and tailoring of the patient's drug treatment regimen. This process facilitates proper medication of patients and also optimizes patient outcomes, ultimately resulting in improved quality of life for the patient while reducing MCO expenditures on the patient's behalf.

A second function for the patient-specific data according to one embodiment of the present invention is to accumulate the data with related information from other patients into an aggregated database for assessing the efficacy of various treatment regimens for specific medical conditions or to identify other health related issues that become more readily apparent when viewed as a collection of multiple medical data versus individual data. This information can be used to continually refine and update the rules and algorithms utilized by the drug therapy analysis engine for assessing patients' therapies and corresponding outcomes. The aggregated information may also be useful for providing feedback to pharmaceutical manufacturers regarding their products. Lastly, the aggregated information can be used to analyze population-based trends and cause-and-effect scenarios, based on patient demographics.

An object of the present invention is to provide a method for generating recommendations for medication therapy for patients. Patient-specific data is generated and transmitted to a processing center. The patient-specific data is aggregated and organized and stored with other data for the patient, and is also added to a knowledge base. The aggregated and organized and stored patient-specific data is then assessed in accordance with a predetermined set of assessment criteria. A report relating to the assessment is generated and then transmitted from the processing center to the patient.

Another object of the present invention is to provide an alternate method for generating recommendations for medication therapy for patients. Patient-specific data is generated, secured, and transmitted to a processing center. The processing center performs a security screening of the patient-specific data in accordance with predetermined screening criteria and accepts only patient-specific data that conforms to the security screening criteria. Accepted patient-specific data is aggregated and organized and stored with other data for the patient, and is also added to a knowledge base. The aggregated and organized patient-specific data is then assessed in accordance with a predetermined set of assessment criteria. A report relating to the assessment is generated and then transmitted from the processing center to the patient.

Yet another object of the present invention is to provide another alternate method for generating recommendations for medication therapy for patients. Patient-specific data is generated, encrypted and transmitted to a processing center. The processing center decrypts the data and aggregates and organizes and stores the data other data for the patient. The data is also added to a knowledge base. The aggregated and organized and stored patient-specific data is then assessed in accordance with a predetermined set of assessment criteria. A report relating to the assessment is generated, encrypted and then transmitted from the processing center to the patient. The report is then decrypted for review by a user.

Yet another object of the present invention is to provide another alternate method for generating recommendations for medication therapy for patients. Patient-specific data is generated, secured, encrypted and transmitted to a processing center. The processing center performs a security screening of the patient-specific data in accordance with predetermined screening criteria and accepts only patient-specific data that conforms to the security screening criteria. The processing center decrypts accepted data and aggregates and organizes and stores the data other data for the patient. The data is also added to a knowledge base. The aggregated and organized and stored patient-specific data is then assessed in accordance with a predetermined set of assessment criteria. A report relating to the assessment is generated, encrypted and then transmitted from the processing center to the patient. The report is then decrypted for review by a user.

Still another object of the present invention is to provide another alternate method for monitoring patient therapy. Patient-specific data is generated for a population of patients and transmitted to a processing center. The patient-specific data is aggregated and organized and stored with other data for the population, forming patient population data. The patient-specific data is also added to a knowledge base. The aggregated and organized patient population data is assessed in accordance with a predetermined set of assessment criteria. A report relating to the assessment is generated and transmitted from the processing center to the patient population.

### BRIEF DESCRIPTION OF THE DRAWINGS

Further features of the inventive embodiments will become apparent to those skilled in the art to which the embodiments relate from reading the specification and claims with reference to the accompanying drawings, in which:

FIG. 1 is a block diagram of a method for monitoring patient therapy according to an embodiment of the present invention;

FIG. 2 is a block diagram of a method for monitoring patient therapy according to an alternate embodiment of the present invention; and

FIG. 3 is a block diagram of a method for monitoring a patient population according to yet another alternate embodiment of the present invention.

### DETAILED DESCRIPTION

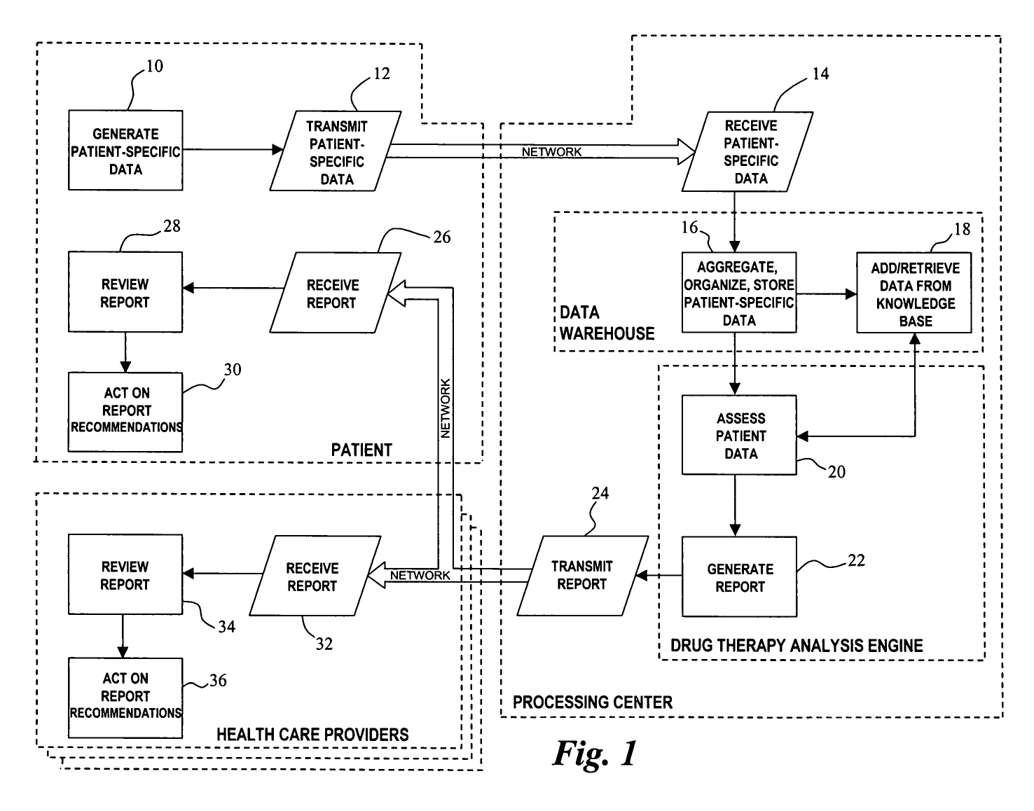

A block diagram of a method for monitoring patient therapy according to an embodiment of the present invention is shown in FIG. 1. At step 10, patient-specific data is generated by the patient or the patient's caregivers, such as family members or the staff of an assisted living facility. Patient-specific data may include patient demographics, clinical and medical history, current medications and therapies, prior medications and therapies, and current physical and mental conditions. A standardized form or set of questions may be utilized to solicit the information in textual form. Further, a set of codes such as the International Classification of Diseases (“ICD”) medical codes or other codes tailored for the present invention may be utilized to document the patient-specific data. The information generated at step 10 is transmitted to a processing center at step 12 via an electronic communication network, such as an intranet or the Intranet or via modem.

The patient-specific data is received by a processing center for the MCO at step 14. The data is sent to a data warehouse where at step 16 it is aggregated and organized and stored with other data for the patient. The data is also added to a “knowledge base” portion of the data warehouse at step 18. A knowledge base is an accumulation of prior experiences and outcomes of other, similarly-situated patients that may be used to predict outcomes and select optimum treatment therapies for current patients suffering from common medical conditions. At step 20 a drug therapy analysis engine assesses the patient's aggregated and organized data in accordance with a predetermined set of assessment criteria, such as the patient's diagnosed conditions and current physical and mental status. The assessment may further be based upon predetermined assessment criteria such as current medical and pharmacological standards and practices as well drawing from the knowledge base of step 18. The assessment at step 20 may be accomplished automatically, for example by means of a predetermined set of instructions or rules and/or algorithms, such as a computer program.

An example scenario of an assessment performed by the drug therapy analysis engine at 20 is a situation where data supplied by the patient or the patient's caregivers indicates that the patient's physical functioning has been deteriorating. The drug therapy analysis engine may detect the change, review the patient's medication records, find that the reduction in physical function occurred subsequent to a change in medication, and determine that a recently prescribed medication, acting in combination with other medications, may be causing an adverse reaction.

After completing the assessment 20, the drug therapy analysis engine generates a report at 22 corresponding to the assessment. The report may include such items as potential adverse side effects and interactions to medications, likelihood of an onset of other symptoms and diseases, and recommendations for alternate therapies. The recommendations may include suggestions for alternate drugs having increased efficacy, lower cost, and fewer side effects. Recommendations may also be made for alternate drugs having improved compatibility when taken in combination with others or the elimination of a previously prescribed medication from the regimen.

The report generated at step 22 is transmitted to the patient at step 24 via the electronic communication network. The patient receives the report at step 26 and reviews it at step 28. The patient may implement the report's recommendations at step 30. Such actions may include changes in diet and personal habits, such as eliminating consumption of alcohol and cigarettes. The patient may also be advised to discuss other recommendations, such as changes in medication, with at least one of the patient's health care providers.

A copy of the report generated at step 22 may also be transmitted directly to the patient's health care providers at step 24. In this case, the patient's health care providers receive the report at step 32, review the report at step 34, implement the report's recommendations at step 36. Such actions may include alterations in the patient's drug therapy regimen.

The drug therapy analysis engine may also assess the patient's data in a trend format to provide early feedback as to the efficacy of any changes in treatment, thereby providing the health care providers with regular cause-and-effect information feedback. In this manner, additional changes in treatment may be implemented if necessary, depending upon the patient's response to previous treatments.

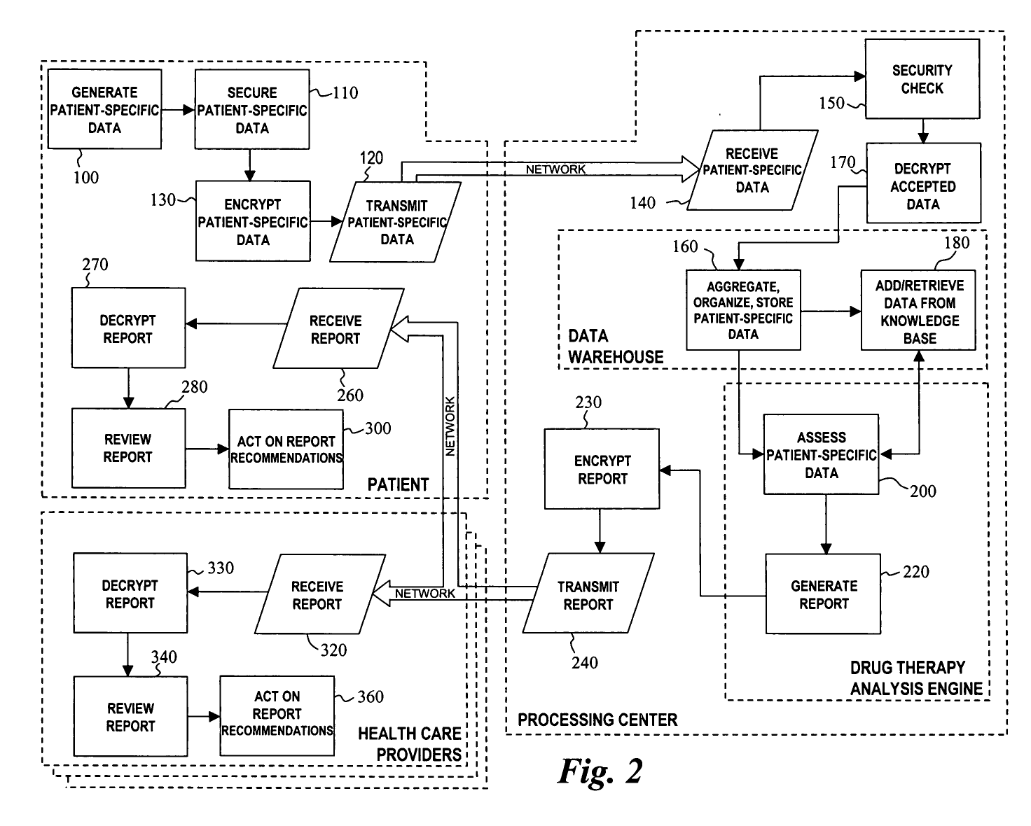

An alternate embodiment of the present invention is depicted in FIG. 2. At step 100, patient-specific data is generated by the patient or the patient's caregivers, such as family members or the staff of an assisted living environment. As previously discussed, patient-specific data may include patient demographics, clinical and medical history, current medications and therapies, prior medications and therapies, and current physical and mental condition. A standardized form or set of questions may be utilized to solicit the information in textual form. Further, a set of codes such as the International Classification of Diseases (“ICD”) medical codes or other codes tailored for the present invention may be utilized to document the patient-specific data.

At step 110, various conventional means of securing the patient-specific data may be utilized to protect patient privacy and prevent tampering. Data security means may include Virtual Private Networks (“VPNs”), validated usernames, and passwords. A conventional “digital signature” or “digital certificate” may also be required to authenticate the user's identity. A digital signature is an electronic signature that can be used to authenticate the identity of the sender of a message or the signer of a document. It can also be used to ensure that the original content of the message or document sent is unchanged. An example digital signature standard is the Digital Signature Standard (“DSS”) established by the National Institute of Standards and Technology (NIST). A digital certificate serves as an electronic “passport” issued by a third party that establishes a user's credentials when doing business or making transactions on electronic communication networks such as the Internet. An example digital certificate standard is the ITU-T X509 international standard established by the International Telecommunications Union (ITU).

At step 130 any conventional form of data encryption may also be used to further protect patient privacy and prevent tampering. Example encryption methods include, without limitation, encryption methods based on the Data Encryption Standard (“DES”) promulgated by NIST and Netscape's Secure Sockets Layer (“SSL”). The secured and encrypted information is then transmitted to a processing center at step 120 via an electronic communication network, such as an intranet or the Intranet or via modem.

The patient-specific data is received by a processing center for the MCO at step 140. The data may be subjected to a security screening at step 150 in accordance with a set of predetermined security screening criteria upon receipt. Security screening criteria may include, without limitation, verification of the data source, a validity check, and a check for computer “viruses.” If the data does not conform to a predetermined portion of the security check, the data may be rejected. In an optional embodiment of the present invention, the sender of the data may be notified of the rejection. Notification may be accomplished by any conventional means, such as an electronic communication network, postal service, telephone and facsimile. If the data is deemed acceptable, it is decrypted at step 170 in a corresponding conventional manner and sent to a data warehouse where at step 160 it is aggregated and organized and stored with other patient-specific data. The accepted data is also added to a knowledge base portion of the data warehouse at step 180. At step 200 a drug therapy analysis engine assesses the patient's aggregated and organized data in accordance with a predetermined set of assessment criteria, such as the patient's diagnosed conditions and current physical and mental status. The assessment may further incorporate additional predetermined assessment criteria, such as current medical and pharmacological standards and practices, as well as drawing from the knowledge base of step 180. The assessment of step 200 may be accomplished automatically, for example by means of a predetermined set of rules and/or algorithms and/or instructions, such as a computer program.

After completing the assessment 200, the drug therapy analysis engine generates a report corresponding to the assessment at step 220. The report may include such items as potential adverse side effects and interactions to medications, likelihood of an onset of other symptoms and diseases, and recommendations. The recommendations may include suggestions for alternate drugs having increased efficacy, lower cost, and fewer side effects. Recommendations may also be made for alternate drugs having improved compatibility when taken in combination with others and/or the removal of one or more medications from the drug treatment regimen.

The report may be encrypted at step 230 in the same manner as at step 130, in order to protect patient privacy. The encrypted report is transmitted to the patient at step 240 via the electronic communication network. The patient receives the report at step 260, decrypts it at step 270, and reviews it at step 280. The patient may implement the report's recommendations at step 300. Such actions may include changes in diet and personal habits, such as eliminating consumption of alcohol and cigarettes. The patient may also be advised to discuss other recommendations, such as changes in medication, with the patient's health care providers.

A copy of the report generated at step 220 may also be transmitted directly to the patient's health care providers at step 240. The health care providers receive the report at step 320, decrypt it at step 330 in the same manner as step 270, review the report at step 340, and may act on the report's recommendations at step 360. Such actions may include alterations in the patient's drug therapy regimen or other treatment.

The drug therapy analysis engine may also assess the patient's data in a trend format to provide early feedback as to the efficacy of any changes in treatment, thereby providing the health care providers with regular cause-and-effect information feedback. In this manner additional changes in treatment may be implemented if necessary, depending upon the patient's response to previous treatments.

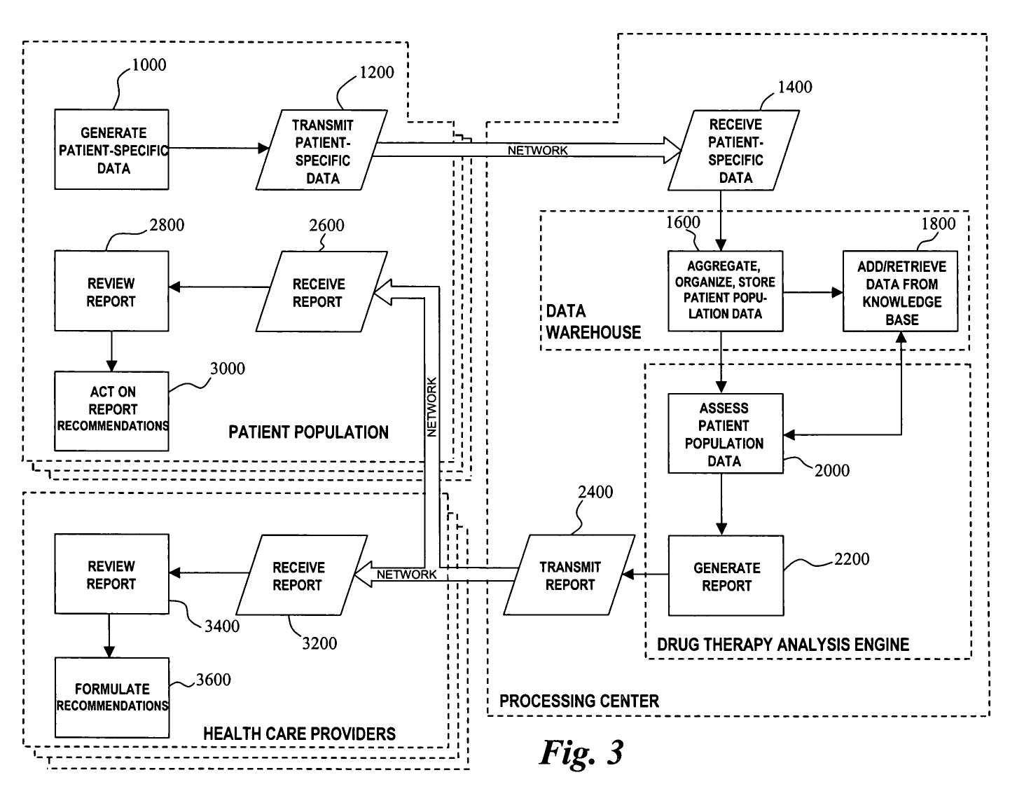

A block diagram of another alternate method for monitoring patient therapy according to an embodiment of the present invention is shown in FIG. 3. In this embodiment of the present invention a patient population comprising a plurality of patients is monitored. Example populations include, but are not limited to, members of a common health care plan, co-employees of a particular company, workers within particular industries, fields of occupations, and patients having particular handicaps or diseases in common.

At step 1000, patient-specific data is generated by a plurality of patients in the population or the patients' caregivers, such as family members, physicians and the staff of an assisted living facility. Patient-specific data may include patient demographics, clinical and medical history, current medications and therapies, prior medications and therapies, and current physical and mental condition. A standardized form or set of questions may be utilized to solicit the information in textual form. Further, a set of codes such as the International Classification of Diseases (“ICD”) medical codes or other codes tailored for the present invention may be utilized to document the patient-specific data. The information generated at step 1000 is transmitted to a processing center at step 1200 via an electronic communication network, such as an intranet or the Intranet or via modem.

The patient-specific data is received by a processing center at step 1400. The data is sent to a data warehouse where at step 1600 it is aggregated and organized and stored with other data for the entire patient population. The data is also added to a knowledge base portion of the data warehouse at step 1800. At step 2000 a drug therapy analysis engine assesses the aggregated and organized patient population data in accordance with a predetermined set of assessment criteria, such as the patients' diagnosed conditions and current physical and mental status. The assessment may further be based upon predetermined assessment criteria such as current medical and pharmacological standards and practices as well drawing from the knowledge base of step 1800. The assessment of step 2000 may be accomplished automatically, for example by means of a predetermined set of instructions and/or rules and/or algorithms, such as a computer program.

After completing the assessment 2000, the drug therapy analysis engine generates a report corresponding to the assessment at step 2200. The report may include such items as potential adverse side effects and interactions to medications, likelihood of an onset of other symptoms and diseases, and recommendations. The recommendations may include suggestions for alternate drugs having increased efficacy, lower cost, and fewer side effects. Recommendations may also be made for alternate drugs having improved compatibility when taken in combination with others.

Step 2200 may further include de-identification of specific patient information to provide for patient privacy and so as to be compliant with patient privacy regulations such as those found in the U.S. Health Insurance Portability and Accountability Act (“HIPAA”). In particular, 45 C.F.R. Parts 160 and 164 of the Act relate to standards for privacy of individually identifiable health information (the “Privacy Rule”), promulgated by the Department of Health and Human Services (HHS). In part the privacy rule can restrict the acquisition and use of certain types of patient-specific data, particularly individually identifiable health information. It should be noted that de-identifying patient-specific data can entail more than merely redacting the patient's name. This is due to the fact that other patient information such as demographics, medical information, and health care facility information could be used in combination to discern the identity of some patients. De-identification can involve the deletion or alteration of some portion of patient data to protect patient privacy, while preserving the overall statistical and analytical integrity of the data. In general, patient-specific medical information may be considered to be de-identified when the following patient information has been removed: name; all geographic subdivisions smaller than a state; complete zip code or equivalents; dates directly related to the patient, ages over 89 or dates indicating such an age; telephone number; fax number; email address; social security number; medical record number; health plan number; account numbers; certificate or license numbers; vehicle identification/serial numbers, including license plate numbers; device identification/serial numbers; Universal Resource Locators (URLs); Internet Protocol (IP) addresses; biometric identifiers; full face photographs and comparable images; and any other patient identifying number, characteristic or code. It is important to note that this list is not exhaustive or complete, and is intended for illustrative purposes only.

The report generated at step 2200 is transmitted to at least a portion of the patients comprising the population at step 2400 via the electronic communication network. The patients receive the report at step 2600 and review it at step 2800. The patients may implement the report's recommendations at step 3000. Such actions may include changes in diet and personal habits, such as eliminating consumption of alcohol and cigarettes. The patients may also be advised to discuss other recommendations, such as changes in medication, with at least one of the patient's health care providers.

A copy of the report generated at step 2200 may also be transmitted directly to the patients' health care providers at step 2400. The patients' health care providers receive the report at step 3200, review the report at step 3400, and decides whether to implement the recommendations of the report at step 3600. Such actions may include, without limitation, alterations in the patients' drug therapy regimen to increase efficacy, reducing treatment costs by switching at least a portion of the patient population's medications to less expensive therapeutic equivalents, changing medication dosages, encouraging beneficial behaviors, and discouraging harmful behaviors and other treatments.

The drug therapy analysis engine may also assess the patient population data in a trend format to provide early feedback as to the efficacy of any changes in treatment, thereby providing the health care providers with regular cause-and-effect information feedback. In this manner additional changes in treatment may be implemented if necessary, depending upon the patients' response to previous treatments.

One skilled in the art will recognize that the aggregated patient population data may be useful to study and compare the medical outcomes of a plurality of patients undergoing the same or similar courses of treatment. Likewise, the patient population data may be used to identify and isolate potential drug-drug interactions, optimize courses of treatment, predict outcomes of similarly-situated patients, and reduce medical costs by favoring courses of treatment having similar efficacy but lower cost. Further, the knowledge base can provide feedback to pharmaceutical manufacturers regarding their products. Lastly, the information can be used to analyze population-based trends and cause-and-effect scenarios, based on patient demographics.

With regard to each of the disclosed embodiments of the present invention, the knowledge base portion of the data warehouse may be used to continually refine and update assessment criteria such as the rules and algorithms utilized by the drug therapy analysis engine for assessing patient outcomes. The aggregated information may also be useful for broader, population-based studies regarding the efficacy of various treatment regimens. Further, the knowledge base can provide feedback to pharmaceutical manufacturers regarding their products. Lastly, the information can be used to analyze population-based trends and cause-and-effect scenarios, based on patient demographics. As previously discussed, access to patient-specific data may be restricted by various forms of protection such as access security, encryption, and de-identification.

The present invention provides a number of improvements in health care. First and foremost, patients benefit from the on-going optimization of the types and amounts of medicines that are administered, maximizing the efficacy of the treatments and in many cases improving the patient's quality of life. The present invention also supplies health care providers with feedback regarding adverse changes in the patient's condition that may be linked to medications. The feedback process is aided by an electronic communication network, which facilitates real-time transfer of data and reports, reducing delays in the delivery and implementation of information. Further, by ensuring that the patients are provided with optimized types and amounts of medications, health care organizations such as MCOs can provide improved health care services while controlling their costs.

While this invention has been shown and described with respect to a detailed embodiment thereof, it will be understood by those skilled in the art that various changes in form and detail thereof may be made without departing from the scope of the claims of the invention.

What is claimed is:

1. A method for generating recommendations for medication therapy for patients, comprising the steps of:
a) generating patient-specific data;
b) transmitting the patient-specific data to a processing center;
c) aggregating and organizing and storing the patient-specific data with other data for the patient;
d) adding the patient-specific data to a knowledge base;
e) assessing the aggregated and organized and stored patient-specific data in accordance with a predetermined set of assessment criteria;
f) generating a report relating to the assessment; and
g) transmitting the report from the processing center to the patient.

2. The method according to claim 1 wherein at least one of the patient-specific data and
report is transmitted by means of an electronic communication network.

3. The method according to claim 1 wherein the assessment criteria further comprises the knowledge base.

4. The method according to claim 1, further comprising the step of transmitting a copy of the report to at least one health care provider.

5. The method according to claim 1 wherein the assessment further comprises a trend analysis of the patient-specific data.

6. The method according to claim 1, further comprising the step of using the patient-specific data to refine the assessment criteria.

7. A method for generating recommendations for medication therapy for patients, comprising the steps of:
a) generating patient-specific data;
b) securing the patient-specific data;
c) transmitting the patient-specific data to a processing center;
d) performing a security screening of the patient-specific data in accordance with a set of predetermined security screening criteria and accepting only the patient-specific data that conforms to the security screening criteria;
e) aggregating and organizing and storing accepted patient-specific data with other data for the patient;
f) adding accepted patient-specific data to a knowledge base;
g) assessing the aggregated and organized and stored patient-specific data in accordance with a predetermined set of assessment criteria;
h) generating a report relating to the assessment; and
h) transmitting the report from the processing center to the patient.

8. The method according to claim 7 wherein at least one of the patient-specific data and report is transmitted by means of an electronic communication network.

9. The method according to claim 7 wherein the assessment criteria further comprises the knowledge base.

10. The method according to claim 7, further comprising the step of transmitting a copy of the report to at least one health care provider.

11. The method according to claim 7 wherein the assessment further comprises a trend analysis of the patient-specific data.

12. The method according to claim 7, further comprising the step of using the patient-specific data to refine the assessment criteria.

13. A method for generating recommendations for medication therapy for patients, comprising the steps of:
a) generating patient-specific data;
b) encrypting the patient-specific data;
c) transmitting the patient-specific data to a processing center;
d) decrypting the patient-specific data at the processing center;
e) aggregating and organizing and storing the patient-specific data with other data for the patient;
f) adding the patient-specific data to a knowledge base;
g) assessing the aggregated and organized and stored patient-specific data in accordance with a predetermined set of assessment criteria;
h) generating a report relating to the assessment;
i) encrypting the report;
j) transmitting the report from the processing center to the patient; and
k) decrypting the report for review by a user.

14. The method according to claim 13 wherein at least one of the patient-specific data and report is transmitted by means of an electronic communication network.

15. The method according to claim 13 wherein the assessment criteria further comprises the knowledge base.

16. The method according to claim 13, further comprising the step of transmitting a copy of the report to at least one health care provider.

17. The method according to claim 13 wherein the assessment further comprises a trend analysis of the patient-specific data.

18. The method according to claim 13, further comprising the step of using the patient-specific data to refine the assessment criteria.

19. A method for generating recommendations for medication therapy for patients, comprising the steps of:
a) generating patient-specific data;
b) securing the patient-specific data;
c) encrypting the patient-specific data;
d) transmitting the patient-specific data to a processing center;
e) performing a security screening of the patient-specific data in accordance with a set of predetermined security screening criteria and accepting only the patient-specific data that conforms to the security screening criteria and rejecting the rest;
f) decrypting accepted patient-specific data at the processing center;
g) aggregating and organizing and storing accepted patient-specific data with other data for the patient;
h) adding accepted patient-specific data to a knowledge base;
i) assessing the aggregated and organized and stored patient-specific data in accordance with a predetermined set of assessment criteria;
j) generating a report relating to the assessment;
j) encrypting the report;
k) transmitting the report from the processing center to the patient; and
l) decrypting the report for review by a user.

20. The method according to claim 19 wherein at least one of the patient-specific data and report is transmitted by means of an electronic communication network.

21. The method according to claim 19 wherein the assessment criteria further comprises the knowledge base.

22. The method according to claim 19, further comprising the step of transmitting a copy of the report to at least one health care provider.

23. The method according to claim 19 wherein the assessment further comprises a trend analysis of the patient-specific data.

24. The method according to claim 19, further comprising the step of using the patient-specific data to refine the assessment criteria.

25. A method for generating recommendations for medication therapy for patients, comprising the steps of:
a) generating patient-specific data for a population of patients;
b) transmitting the patient-specific data to a processing center;
c) aggregating and organizing and storing the patient-specific data with other data for the population, forming patient population data;
d) adding the patient-specific data to a knowledge base;
e) assessing the aggregated and organized patient population data in accordance with a predetermined set of assessment criteria;
f) generating a report relating to the assessment; and
g) transmitting the report from the processing center to the patient population.

26. The method according to claim 25 wherein at least one of the patient-specific data and report are transmitted by means of an electronic communication network.

27. The method according to claim 25 wherein the assessment criteria further comprises the knowledge base.

28. The method according to claim 25, further comprising the step of transmitting a copy of the report to at least one health care provider.

29. The method according to claim 25 wherein the assessment further comprises a trend analysis of the patient population data.

30. The method according to claim 25, further comprising the step of using the patient population data to refine the assessment criteria.

31. The method according to claim 25, wherein the step of generating a report further comprises de-identifying patient-specific data.
